(19) United States
(12) Patent Application Publication
(10) Pub. No.: US 2014/0128753 A1
(43) Pub. Date: May 8, 2014

(54) PIEZOELECTRIC HEART RATE SENSING FOR WEARABLE DEVICES OR MOBILE DEVICES

(71) Applicants: Michael Edward Smith Luna, San Jose, CA (US); Scott Fullam, Palo Alto, CA (US)

(72) Inventors: Michael Edward Smith Luna, San Jose, CA (US); Scott Fullam, Palo Alto, CA (US)

(73) Assignee: AliphCom, San Francisco, CA (US)

(21) Appl. No.: 13/672,398

(22) Filed: Nov. 8, 2012

(51) Int. Cl. A61B 5/024; A61B 5/00; A61B 7/04

(52) U.S. Cl. CPC A61B 5/02438; A61B 7/04; A61B 5/0004; A61B 5/7278; A61B 5/6831
USPC 600/500; 600/528

(57) ABSTRACT

Embodiments relate generally to electrical and electronic hardware, computer software, wired and wireless network communications, and wearable computing devices for sensing health and wellness-related information. More specifically, disclosed is a physiological sensor using, for example, acoustic signal energy to determine physiological characteristics, such as a heart rate, the physiological sensor being disposed in a wearable device (or carried device). In one embodiment, a physiological signal generator is disposed substantially in a wearable housing. At least a portion of a skin surface microphone (“SSM”) including a piezoelectric sensor is configured to receive acoustic signals. The wearable housing is configured to position the SSM to receive an acoustic signal originating from human tissue. The physiological signal generator is configured to receive a piezoelectric signal based on an acoustic signal, and to generate a physiological signal including data representing a heartbeat or heart rate.

### FIELD

Embodiments relate generally to electrical and electronic hardware, computer software, wired and wireless network communications, and wearable computing devices for sensing health and wellness-related physiological characteristics. More specifically, disclosed is a physiological sensor using, for example, acoustic signal energy to determine physiological characteristics, such as a heart rate, the physiological sensor being disposed in a wearable device (or carried device).

### BACKGROUND

Devices and techniques to gather physiological information, such as a heart rate of a person, while often readily available, are not well-suited to capture such information other than by using conventional data capture devices. Conventional devices typically lack capabilities to capture, analyze, communicate, or use physiological-related data in a contextually-meaningful, comprehensive, and efficient manner, such as during the day-to-day activities of a user, including high impact and strenuous exercising or participation in sports. Further, traditional devices and solutions to obtaining physiological information, such as heart rate, generally require that the sensors remain firmly affixed to the person to employ, for example, low-level electrical signals (i.e., Electrocardiogram (“ECG”) signals). In some conventional approaches, a few sensors are placed directly on the skin of a person while the sensors and the person are to remain relatively stationary during the measurement process. While functional, the traditional devices and solutions to collecting physiological information are not well-suited for use during the course of one's various life activities, nor are traditional devices and solutions well-suited for active participants in sports or over the course of one or more days.

Thus, what is needed is a solution for data capture devices, such as for wearable devices, without the limitations of conventional techniques.

### BRIEF DESCRIPTION OF THE DRAWINGS

Various embodiments or examples (“examples”) of the invention are disclosed in the following detailed description and the accompanying drawings:

FIG. 1 illustrates an example of a physiological sensing device disposed in a wearable data-capable band, according to some embodiments;

FIG. 2A is a diagram depicting examples of positions at which a piezoelectric sensor can be disposed, according to some examples;

FIG. 2B is a diagram depicting examples of devices in which a heart rate signal generator and a piezoelectric sensor, and their components, can be disposed or distributed among, according to some examples;

FIGS. 3A to 3C depict a wearable device including a piezoelectric sensor in various configurations, according to some embodiments;

FIG. 4 depicts an example of a heart rate signal generator, according to some embodiments;

FIG. 5 depicts an example of filtering anomalous heartbeat signals, according to some embodiments;

FIG. 6 is an example flow diagram for sensing heart rate, according to some embodiments; and

FIG. 7 illustrates an exemplary computing platform disposed in a wearable device in accordance with various embodiments.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

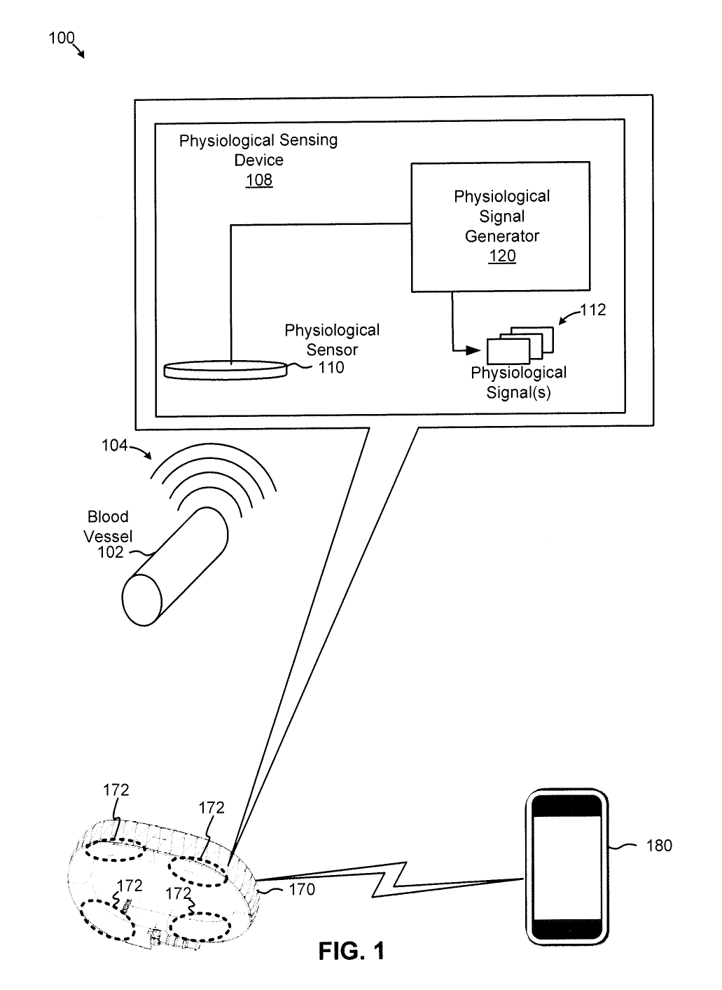

FIG. 1 illustrates an example of a physiological sensing device disposed in a wearable data-capable band, according to some embodiments. Diagram 100 depicts physiological sensing device 108 configured to generate one or more physiological signals, such as heart rate, respiration, and other detectable physiological characteristics, for example, of a wearer of a wearable device in which physiological sensing device 108 is disposed or otherwise associated. Physiological sensing device 108 includes a physiological sensor 110 and a physiological signal generator 120. Physiological sensor 110 is configured to sense signals, such as physiological signals, associated with a physiological characteristic. As such, physiological sensor 110 can be disposed adjacent to a source of physiological signals, such as adjacent to a blood vessel 102. According to some embodiments, physiological sensor 110 is a piezoelectric sensor (e.g., a piezoelectric transducer) configured to receive, for example, acoustic energy, and further configured to generate piezoelectric signals (e.g., electrical signals). In the example shown, piezoelectric sensor 110 is configured to receive acoustic signal 104 that includes heart-related information. For example, acoustic signal 104 can propagate through at least human tissue as one or more sound energy waveforms. Such sound energy signals can originate from either a beating heart (e.g., via a blood vessel 102) or blood pulsing through blood vessel 102, or both. The energy that propagates as acoustic signal 104 into piezoelectric sensor 110 is converted into piezoelectric signals, which, in turn, are transmitted to physiological signal generator 120. In some embodiments, piezoelectric sensor 110 forms either a skin surface microphone (“SSM”) or a portion thereof. An SSM is configured to receive acoustic energy originating from human tissue rather than airborne acoustic sources that otherwise produce acoustic energy waveforms to propagate through the medium of air.

Physiological signal generator 120 is configured to detect and identify, for example, heartbeats, and is further configured to generate physiological signals 112, such as a heart rate signal or any other signal including data describing one or more physiological characteristics associated with a user that is wearing or carrying physiological sensing device 108. In some examples, a heart rate signal or other physiological signals, can be determined (i.e., recovered) from the measured acoustic signals by, for example, comparing the measured acoustic signal against data associated with one or more waveforms of candidate heartbeats. For example, physiological signal generator 120 can compare, for example, the magnitude of acoustic signal 104 over time against profiles defining characteristics of candidate heartbeats to identify a heartbeat. A profile can be a data files that defines, describes or otherwise includes characteristics of heartbeats (e.g., in terms of magnitude, timing, pattern reoccurrence, etc.) against which measured data can be compared to determine whether a capture signal portion relates to a heartbeat, according to some embodiments.

In some embodiments, physiological sensing device 108 can be disposed in a wearable device 170, and piezoelectric sensor 110 can be disposed at approximate portions 172 of wearable device 170. In some cases, piezoelectric sensor 110 approximate portions 172 are more likely to be adjacent a radial or ulnar artery than other portions. In some instances, approximate portions 172 provide relatively shorter distances through which acoustic signals propagate from a source to piezoelectric sensor 110. Further, the housing of wearable device 170 can encapsulate, or substantially encapsulate, piezoelectric sensor 110. Thus, piezoelectric sensor 110 can have a portion that is disposed external to the housing of wearable device 170 to contact a skin of a wearer. Or, piezoelectric sensor 110 can be disposed in wearable device 170, which can be formed, at least partially, using an encapsulant that has an acoustic impedance that is equivalent to or is substantially similar to that of human tissue. While wearable device 170 is shown to have an elliptical-like shape, it is not limited to such a shape and can have any shape. Note that physiological sensing device 108 is not limited to being disposed adjacent blood vessel 102 in an arm, but can be disposed on any portion of a user's person (e.g., on an ankle, ear lobe, behind an ear, around a finger or on a fingertip, etc.).

In view of the foregoing, the functions and/or structures of piezoelectric sensor 110 and physiological information generator 120, as well as their components, can facilitate the sensing of physiological characteristics, including heart rate, in situ during which a user is engaged in physical activity. With the use of piezoelectric sensors as described herein, electrical signals need not be sensed in human tissue as can be the case in ECG monitoring and bioimpedance sensing. Thus, sensing bio-electric signals need not be at issue when considering proximity to the source of physiological characteristic. Piezoelectric sensor 110 can be used to sense via acoustic signal 104 as a heart-related signal. At least in some instances, the acoustic energy of heart-related signals can propagate through human tissue and/or a vascular system for relatively lengthy distances (e.g., through a limb or the body generally).

In some embodiments, physiological sensor 110 can any suitable structure and sensor for picking up and transferring signals, regardless of whether the signals are electrical, magnetic, optical, pressure-based, physical, acoustic, etc., according to various embodiments. According to some embodiments, physiological sensor 110 can be configured to couple acoustically to a target location or by other means associated with the type of sensor used.

Piezoelectric sensor 110 can form a skin surface microphone (“SSM”), or a portion thereof, according to some embodiments. An SSM can be an acoustic microphone configured to enable it to respond to acoustic energy originating from human tissue rather than airborne acoustic sources. As such, an SSM facilitates relatively accurate detection of physiological signals through a medium for which the SSM can be adapted (e.g., relative to the acoustic impedance of human tissue). Examples of SSM structures in which piezoelectric sensors can be implemented (e.g., rather than a diaphragm) are described in U.S. patent application Ser. No. 11/199,856, filed on Aug. 8, 2005. As used herein, the term human tissue can refer to, at least in some examples, as skin, muscle, blood, or other tissue. In some embodiments, a piezoelectric sensor can constitute an SSM.

In some embodiments, wearable device 170 can be in communication (e.g., wired or wirelessly) with a mobile device 180, such as a mobile phone or computing device. In some cases, mobile device 180, or any networked computing device (not shown) in communication with wearable device 170 or mobile device 180, can provide at least some of the structures and/or functions of any of the features described herein. As depicted in FIG. 1 and subsequent figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1 (or any subsequent figure) can represent one or more algorithms. Or, at least one of the elements can represent a portion of logic including a portion of hardware configured to provide constituent structures and/or functionalities.

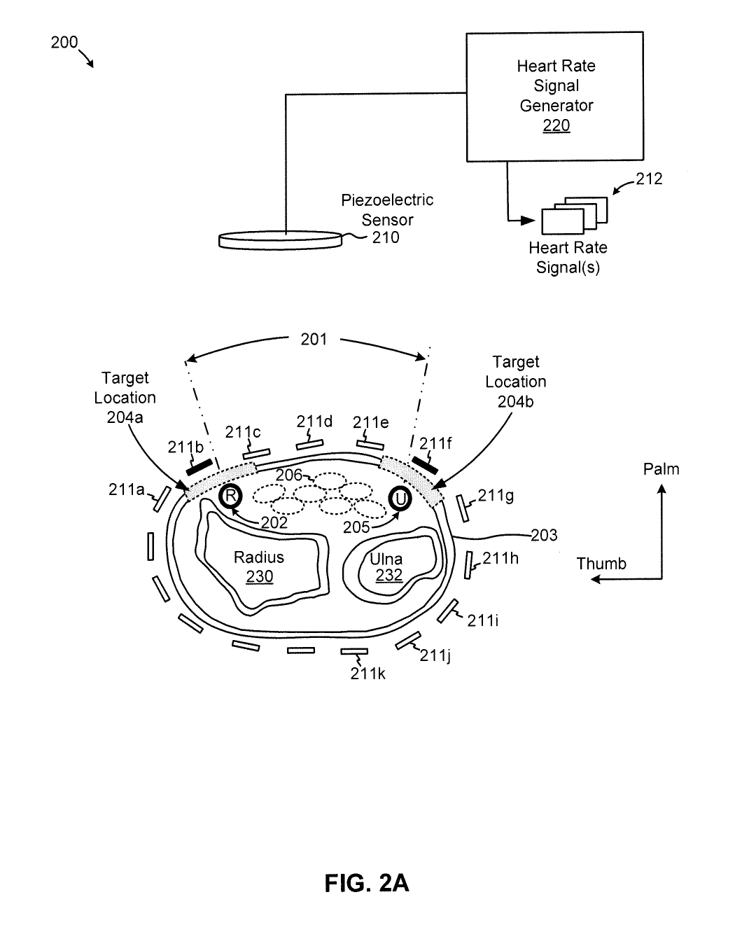

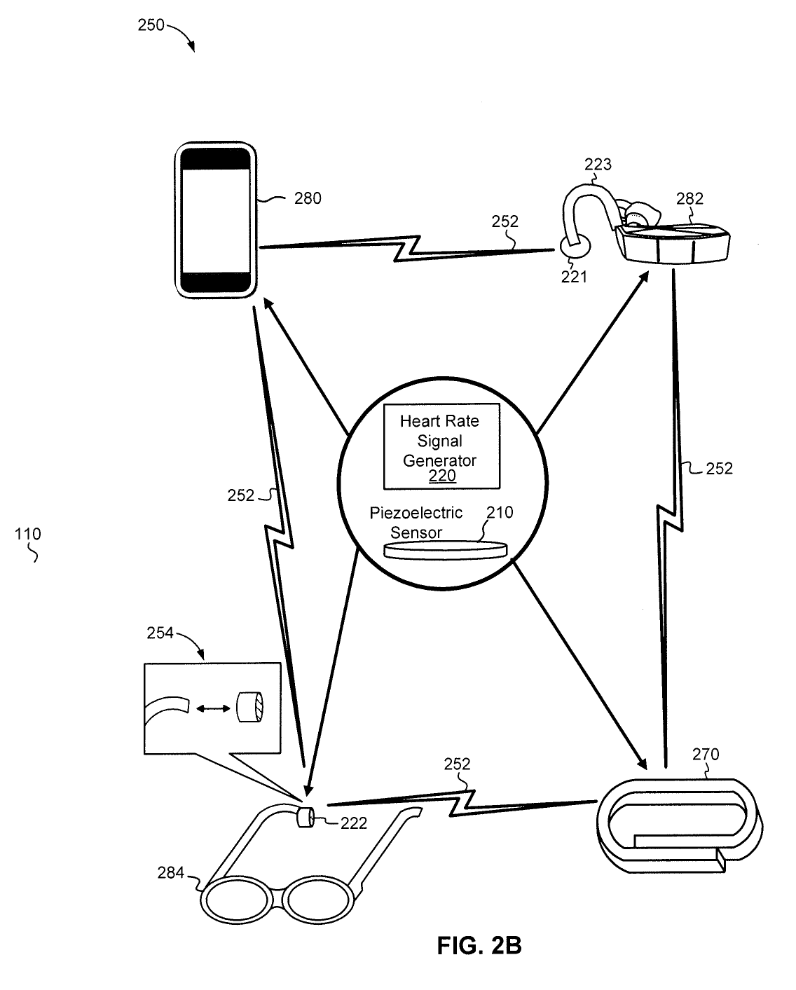

FIG. 2A is a diagram depicting examples of positions at which a piezoelectric sensor can be disposed, according to some examples. Diagram 200 depicts a heart rate signal generator 220 configured to generate heart rate signals 212 specifying a heart rate for a user. Heart rate signal generator 220 generates heart rate signals 212 based on piezoelectric signals received from piezoelectric sensor 210. Diagram 200 further depicts positions at which piezoelectric sensor 210 may be placed. In particular, positions 211a to 211k represent positions at which piezoelectric sensor 210 can be disposed in a wearable device worn on or about a wrist 203 of a user.

In the cross-sectional view shown in FIG. 2A, positions 211a, 211b, 211c, 211d, 211e, 211f, 211g, 211h, 211i, 211j, and 211k, among others, describe positions at which piezoelectric sensor 210 can be disposed about wrist 203 (or the forearm). The cross-sectional view of wrist 203 also depicts a radius bone 230, an ulna bone 232, flexor muscles/ligaments 206, a radial artery (“R”) 202, and an ulna artery (“U”) 205. Radial artery 202 is at a distance 201 (regardless of whether linear or angular) from ulna artery 205. Distance 201 may be different, on average, for different genders, based on male and female anatomical structures. In some cases, piezoelectric sensor 210 (and/or the ability of acoustic signals to propagate through human tissue) can obviate a requirement for a specific placement of piezoelectric sensor 210 due to different anatomical structures based on gender, preference of the wearer, or any other issue that affects placement of piezoelectric sensor 210 that otherwise may not be optimal.

According to some embodiments, target locations 204a and 204b represent optimal areas (or volumes) at which to measure, monitor and capture data related to acoustic physiological signals. In particular, target location 204a represents an optimal area adjacent radial artery 202 to pick up acoustic signals, whereas target location 204b represents another optimal area adjacent ulna artery 205 to pick up other acoustic signals. For example, positions 211b and 211f can receive acoustic signals associated with radial artery 202 and ulna artery 205, respectively without intervening tissues masses, such as flexor muscles/ligaments 206 or bones 230 and 232. As used herein, the term “target location” can, for example, refer to a region in space from which a physiological characteristic can be determined. A target region can be adjacent to a source of a physiological characteristic, such as blood vessel 102, with which an acoustic signal can be captured and analyzed to identify one or more physiological characteristics. The target region can reside in two-dimensional space, such as an area on the skin of a user adjacent to the source of the physiological characteristic, or in three-dimensional space, such as a volume that includes the source of the physiological characteristic. More or fewer piezoelectric sensors 210 can be used.

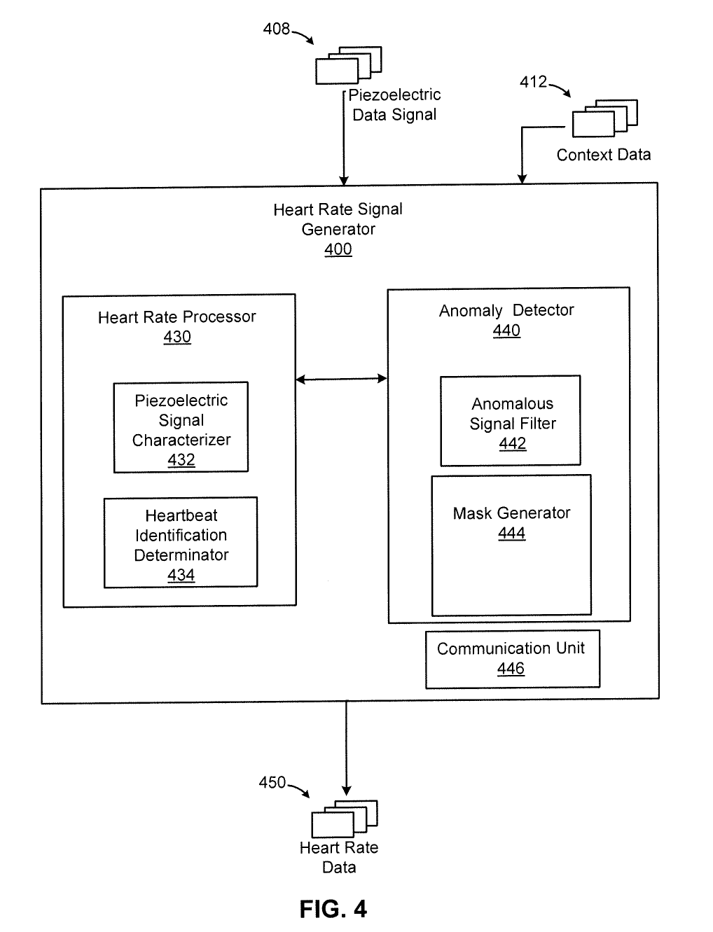

FIG. 2B is a diagram depicting examples of devices in which a heart rate signal generator and a piezoelectric sensor, and their components, can be disposed or distributed among, according to some examples. Diagram 250 depicts examples of devices (e.g., wearable or carried) in which heart rate signal generator 220 and piezoelectric sensor 210 can be disposed include, but are not limited to, a mobile phone 280, a headset 282, eyewear 284, and a wrist-based wearable device 270. In some instances, heart rate signal generator 220 and/or piezoelectric sensor 210 can be implemented as an acoustic heart rate sensor 221 or 222. Acoustic heart rate sensor 221 is disposed on or at an earloop 223 of headset 282 (e.g., a Wi-Fi or Bluetooth® communications headset) to position piezoelectric sensor 210 adjacent to human tissue (e.g., behind an ear). Acoustic heart rate sensor 222 is disposed on or at the ends of eyewear 284 (e.g., at temple tips that extend over an ear) to position piezoelectric sensor 210 adjacent to human tissue (e.g., behind an ear). Acoustic heart rate sensors, such as sensor 222, can be configured to detach and attach, as shown in view 254, to any of the devices described. Further, acoustic heart rate sensors described in FIG. 2B can include a communications unit, such as described in FIG. 4, to establish communications links 252 (e.g., wireless or acoustic data links) to communicate heart-related data signals among the devices.

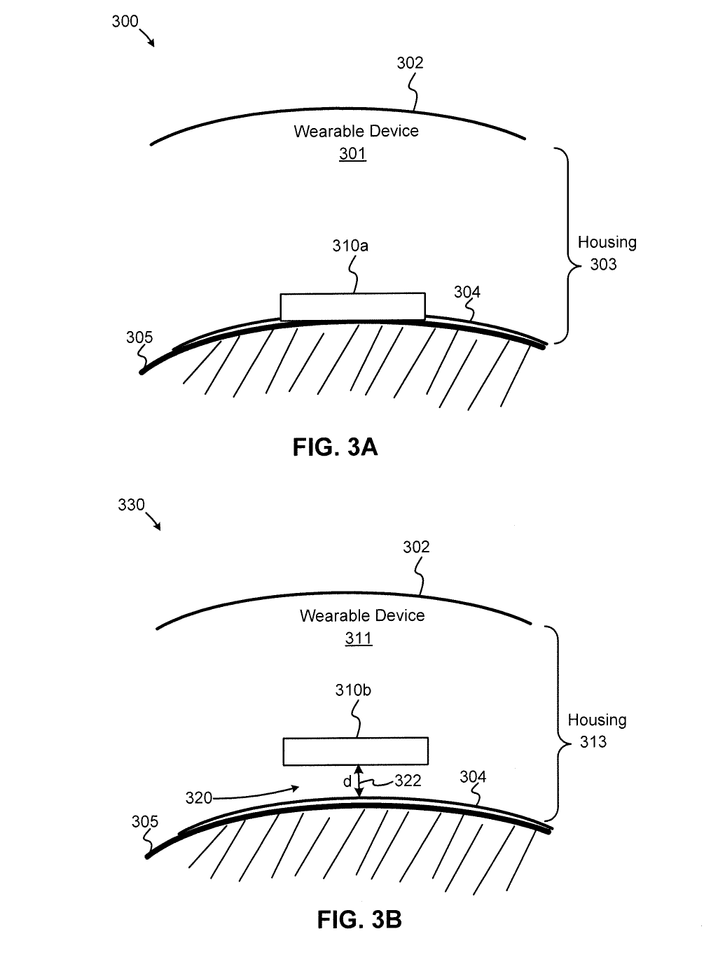

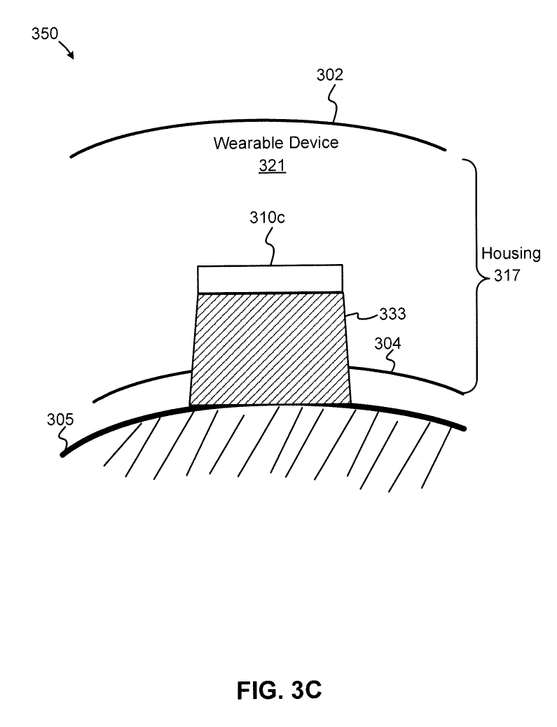

FIGS. 3A to 3C depict a wearable device including a piezoelectric sensor in various configurations, according to some embodiments. Diagram 300 of FIG. 3A depicts a wearable device 301, which has an outer surface 302 and an inner surface 304. In some embodiments, wearable device 301 includes a housing 303 configured to position a piezoelectric sensor 310a (or an SSM including a piezoelectric sensor) to receive an acoustic signal originating from human tissue, such as skin surface 305. As shown, at least a portion of piezoelectric sensor 310a is formed external to surface 304 of wearable housing 303. The exposed portion of the piezoelectric sensor is configured to contact skin 305.

Diagram 330 of FIG. 3B depicts a wearable device 311, which has an outer surface 302 and an inner surface 304. In some embodiments, wearable device 311 includes a housing 313 configured to position a piezoelectric sensor 310b (or an SSM including a piezoelectric sensor) to receive an acoustic signal originating from human tissue, such as skin surface 305. As shown, piezoelectric sensor 310b is disposed in wearable housing 313 at a distance (“d”) 322 from inner surface 304. Material, such as an encapsulant, can be used to form wearable housing 313 to reduce or eliminate exposure to elements in the environment external to wearable device 311. In some embodiments, a portion of an encapsulant or any other material can be disposed or otherwise formed at region 320 to facilitate propagation of an acoustic signal to the piezoelectric sensor. The material and/or encapsulant can have an acoustic impedance value that matches or substantially matches the acoustic impedance of human tissue and/or skin. Values of acoustic impedance of the material and/or encapsulant can be described as being substantially similar to the human tissue and/or skin when the acoustic impedance of the material and/or encapsulant varies no more than 60% of that of human tissue or skin, according to some embodiments.

Examples of materials having acoustic impedances matching or substantially matching the impedance of human tissue can have acoustic impedance values in a range that includes 1.5×10⁶ Pa×s/m (e.g., an approximate acoustic impedance of skin). In some examples, materials having acoustic impedances matching or substantially matching the impedance of human tissue can provide for a range between 1.0×10⁶ Pa×s/m and 1.0×10⁷ Pa×s/m. Note that other values of acoustic impedance can be implemented to form one or portions of housing 313. In some examples, the material and/or encapsulant can be formed to include at least one of silicone gel, dielectric gel, thermoplastic elastomers (TPE), and rubber compounds, but is not so limited. As an example, the housing can be formed using Kraiburg TPE products. As another example, housing can be formed using Sylgard® Silicone products. Other materials can also be used.

Diagram 350 of FIG. 3C depicts a wearable device 321, which has an outer surface 302 and an inner surface 304. In some embodiments, wearable device 321 includes a housing 317 configured to position a piezoelectric sensor 310c (or an SSM including a piezoelectric sensor) to receive an acoustic signal originating from human tissue, such as skin surface 305. A portion of the piezoelectric sensor is configured to receive acoustic signals via a coupler 333 from skin 305. As shown, piezoelectric sensor 310c is disposed in wearable housing 313 at a distance from inner surface 304. In this example, coupler 333 is disposed between piezoelectric sensor 310c and inner surface 304 and is configured to contact skin 305 at one end and to communicate acoustic signals to piezoelectric sensor 310c at the other end. Coupler 333 can be composed of an equivalent material to that described in FIG. 3B to facilitate propagation of an acoustic signal to piezoelectric sensor 310c.

FIG. 4 depicts an example of a heart rate signal generator, according to some embodiments. The diagram of FIG. 4 depicts a heart rate signal generator 400 that can be disposed in a wearable device or distributed over the wearable device and other devices, such as a mobile computing device or phone. Heart rate signal generator 400 can be configured to receive piezoelectric data signals 408 from a piezoelectric sensor and, optionally, context data 412. Context data 412 includes data describing the context in which a heart rate is being determined. For example, context data 412 includes an age of the user, motion data describing an activity or general level of motion of the user (e.g., whether the user is sleeping, sitting, running a marathon, etc.), a location of the user, and other types of data that can assist determining a heart rate. The age of the user can determine normative or expected heart rates as older users typically have slow heart rates than younger users. This information can assist in excluding anomalous data. Heart rate signal generator 400 also can be configured to generate heart rate data 450 that describes the heart rate of a user.

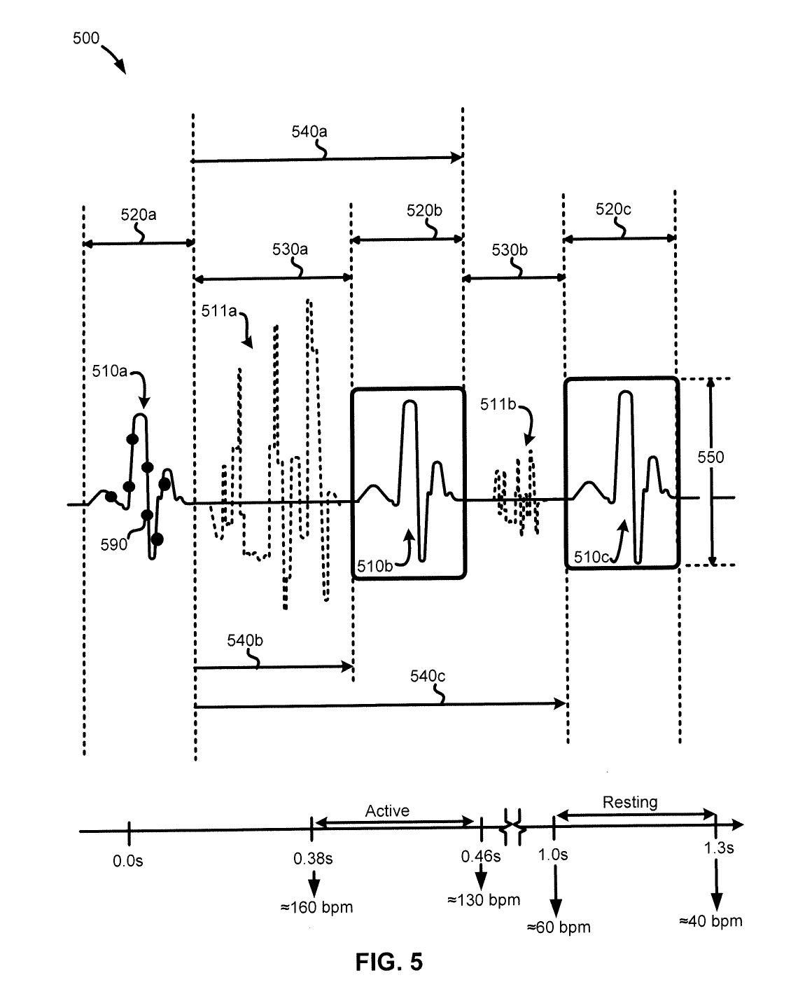

Heart rate signal generator 400 can include one or more of a heart rate processor 430 configured to determine one or more heartbeats constituting a heart rate, and an anomaly detector 440 configured to detect or otherwise exclude data that are unlikely related to a heartbeat. As used herein, the term anomalous data or signals can refer, at least in some examples, to data and/signals that have values that may be inconsistent with expected values describing a range of values associated with candidate heart beats. For example, a candidate heartbeat, such as heart beat 510a of FIG. 5, can be described in terms of one or more data points 590 of FIG. 5 expressing detected signal magnitudes at different times. As a candidate heartbeat, data points 590 (e.g., samples) can represent likely heartbeat characteristics (e.g., magnitudes and timing) that can define expected data points and characteristics of likely heartbeats. These characteristics, when analyzed within certain tolerances, can indicate whether piezoelectric data signals 408 (or portions thereof) indicate a heartbeat, when compared to piezoelectric data signals 408. Referring back to FIG. 4, heart rate processor 430 is configured to compare measured portions of piezoelectric data signal 408 to data files (e.g., profiles) that define characteristics of heartbeats (e.g., in terms of magnitude, timing, pattern reoccurrence, etc.), according to some embodiments.

Heart rate processor 430 can include a piezoelectric signal characterizer 432 and a heartbeat identification determinator 434. Piezoelectric signal characterizer 432 is configured to amplify the piezoelectric data signals and to characterize the values of piezoelectric data signals 408. For example, piezoelectric signal characterizer 432 can determine characteristics of portions of piezoelectric signals to, for example, establish values associated with data points, such as data points 590 of FIG. 5.

Anomaly detector 440 can include an anomalous signal filter 442 and a mask generator 444. Anomalous signal filter 442 is configured to determine which data points 590 (or samples) are considered valid for purposes of determining a heartbeat. For example, data points having magnitudes above an expected magnitude of an acoustic signal generated by a heart-related event likely are not due to pulsing blood (e.g., it is rare that a sudden, instantaneous exertion of the heart occurs). Thus, anomalous signal filter 442 can indicate that data points 590 above a certain magnitude ought not be considered as part of a heartbeat. In some implementations, anomalous signal filter 442 receives the characterized piezoelectric signals from piezoelectric signal characterizer 432.

Mask generator 444 is also configured to mask or otherwise exclude data from heartbeat consideration when determining one or more heartbeats. Mask generator 444 consumes context data 412. For example, older users and younger users are expected to have different heart rates when resting and being active. As such, mask generator 444 excludes from consideration heart rates that occur in other age ranges that need not pertain to the age range in which the user occupies. As another example, mask generator 444 excludes from consideration heart rates that are inconsistent with motion data (e.g., a high heart rate range of 130 to 160 bpm is excluded if motion data suggests that the person is resting or sleeping). Likewise, changes in location due to user-generated motion (e.g., running) is unlikely to be accompanied by heart rates indicative to sleeping. Therefore, mask generator 444 excludes from consideration heart rates that are below those that define an active person, when, in fact, the user is in motion. Further, mask generator 444 can define windows or intervals within to analyze a next heart beat based on previous samples of heartbeats. As heart rates to do not normally change instantaneously, mask generator 444 can modify the timing when the windows or intervals open to accept data presumed valid and when to exclude other data unlikely to be heart-related. Mask generator 444 is configured to provide heartbeat identification determinator 434 with piezoelectric data samples that have not been masked, whereby heartbeat identification determinator 434 determines a heartbeat and an approximate point in time at which the heart beat occurs. Subsequent heartbeats can be determined relative to the point in time in which an earlier heart beat has been determined. Heartbeat identification determinator 434 can then generate heart rate data 450 that includes a real-time (or near real-time) heart rate. In some embodiments, heart rate signal generator 400 can include a communication unit 446 including hardware, software, or a combination thereof, configured to transmit and receive control and heart-related data to other devices, such as those described in FIG. 2. Heart rate signal generator 400 and/or anomaly detector 440 can operate individually or cooperatively to determine trend data representing approximate intervals between heartbeats over time. The approximate intervals can change as the user transitions through different levels of activity (e.g., from resting to walking to running).

FIG. 5 depicts an example of filtering anomalous heartbeat signals, according to some embodiments. Diagram 500 of FIG. 5 depicts portions of a piezoelectric signal including portions 510a, 510b, and 510c that include characteristics that predominantly match those of expected heartbeats. In this example, consider that portion 510a is determined to include or represent a valid representation of a heartbeat during interval 520a. In some examples, portion 511a is determined to include amplitudes or magnitudes that exceed an expected magnitude 550. Therefore, anomaly detector 440 can invalidate or mask portion 511a from being considered. Further, portion 511b is determined to include amplitudes or magnitudes that fall below an expected minimum magnitude (not shown), and can be invalidated to remove from consideration. Alternatively, or in addition to the aforementioned, portion 511a can be determine to coincide with interval 530a (e.g., above 160 bpm), and thus can be invalidated (and masked). Portion 511b can occur during intervals 530b, which can be either slower than during active interval 520b or faster than during resting interval 520c. Thus, portion 511b can be invalidated (and masked) if the user's activity does not suggest a heart rate associate with the timing of portion 511b. Mask generator 444 can be further configured to exclude portion 510c when a trend of heartbeat data suggest that the window in which to accept data is from time 540b to time 540a after a heartbeat is detected at 520a (i.e., the user is active). Or, mask generator 444 can be further configured to exclude portion 510b when a trend of heartbeat data suggests that the window in which to accept data is during 520c after a time 540c when heartbeat is detected at 520a (i.e., the user is resting).

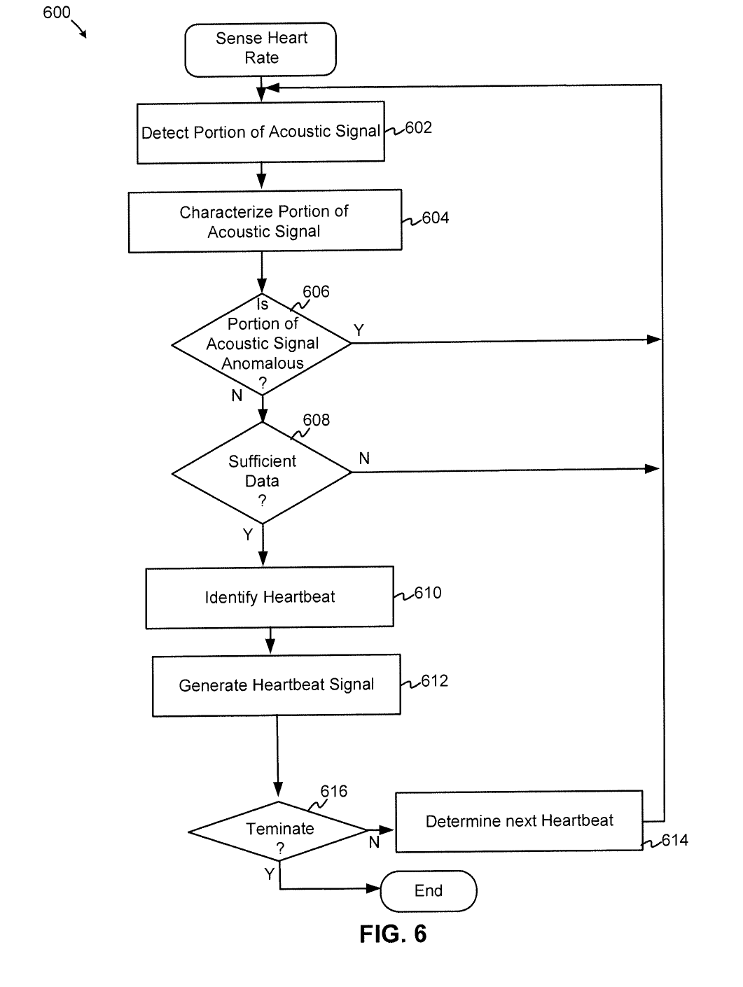

FIG. 6 is an example flow diagram for sensing heart rate, according to some embodiments. At 602, flow 600 detects a portion of an acoustic signal (e.g., as a piezoelectric signal portion). At 604, one or more portions of the acoustic signal are characterized to determine whether the portions include hear-related signals. At 606, a determination is made as to whether a portion is anomalous. If so, that portion is excluded from consideration, and flow 600 moves to 602. Otherwise, flow 600 moves to 608 to determine whether sufficient data is obtained to make a determination whether a portion of an acoustic signal can be deemed a heartbeat. If not, flow 600 moves to 602 to determine additional samples, otherwise flow 660 moves to 610 at which a heartbeat is identified. At 612, a heartbeat signal is generated including information about a heart rate. If flow 600 is to terminate, it does so at 616. Otherwise, a next heartbeat is determined at 614 as flow 600 continues.

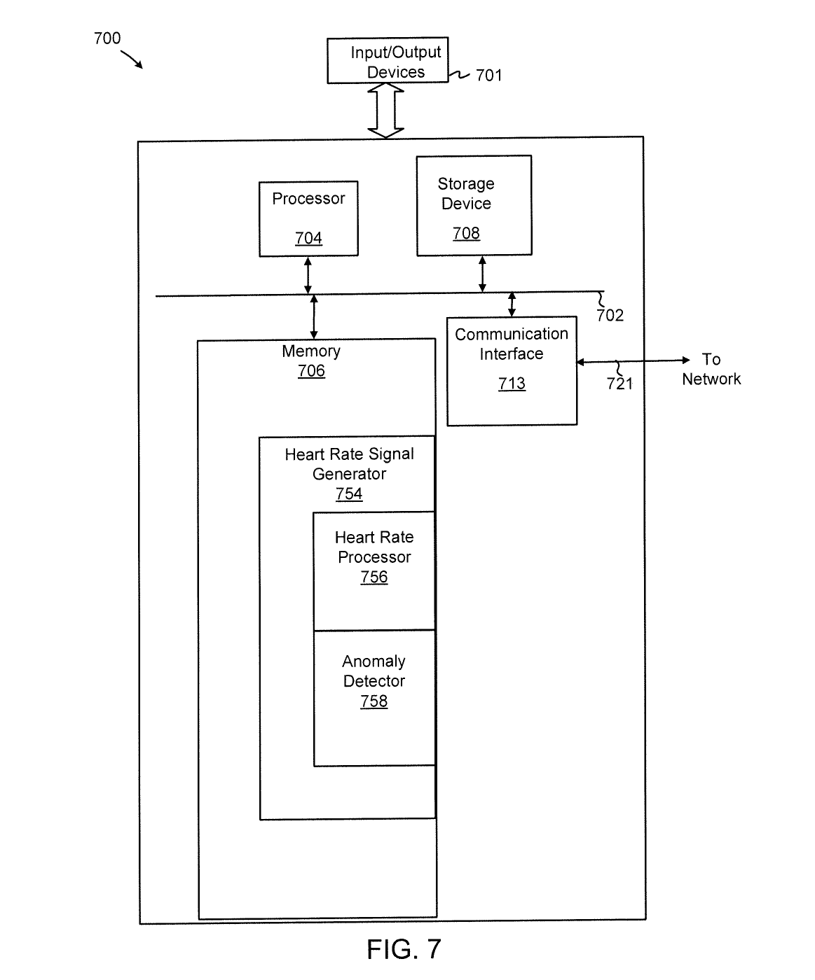

FIG. 7 illustrates an exemplary computing platform disposed in a wearable device in accordance with various embodiments. In some examples, computing platform 700 may be used to implement computer programs, applications, methods, processes, algorithms, or other software to perform the above-described techniques. Computing platform 700 includes a bus 702 or other communication mechanism for communicating information, which interconnects subsystems and devices, such as one or more processors 704, system memory 706 (e.g., RAM, etc.), storage device 708 (e.g., ROM, etc.), a communication interface 713 (e.g., an Ethernet or wireless controller, a Bluetooth controller, etc.) to facilitate communications via a port on communication link 721 to communicate, for example, with a computing device, including mobile computing and/or communication devices with processors. Processor 704 can be implemented with one or more central processing units (“CPUs”), such as those manufactured by Intel® Corporation, or one or more virtual processors, as well as any combination of CPUs and virtual processors. Computing platform 700 exchanges data representing inputs and outputs via input-and-output devices 701, including, but not limited to, keyboards, mice, audio inputs (e.g., speech-to-text devices), user interfaces, displays, monitors, cursors, touch-sensitive displays, LCD or LED displays, and other I/O-related devices.

According to some examples, computing platform 700 performs specific operations by processor 704 executing one or more sequences of one or more instructions stored in system memory 706, and computing platform 700 can be implemented in a client-server arrangement, peer-to-peer arrangement, or as any mobile computing device, including smart phones and the like. Such instructions or data may be read into system memory 706 from another computer readable medium, such as storage device 708. In some examples, hard-wired circuitry may be used in place of or in combination with software instructions for implementation. Instructions may be embedded in software or firmware. The term “computer readable medium” refers to any tangible medium that participates in providing instructions to processor 704 for execution. Such a medium may take many forms, including but not limited to, non-volatile media and volatile media. Non-volatile media includes, for example, optical or magnetic disks and the like. Volatile media includes dynamic memory, such as system memory 706.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 702 for transmitting a computer data signal.

In some examples, execution of the sequences of instructions may be performed by computing platform 700. According to some examples, computing platform 700 can be coupled by communication link 721 (e.g., a wired network, such as LAN, PSTN, or any wireless network) to any other processor to perform the sequence of instructions in coordination with (or asynchronous to) one another. Computing platform 700 may transmit and receive messages, data, and instructions, including program code (e.g., application code) through communication link 721 and communication interface 713. Received program code may be executed by processor 704 as it is received, and/or stored in memory 706 or other non-volatile storage for later execution.

In the example shown, system memory 706 can include various modules that include executable instructions to implement functionalities described herein. In the example shown, system memory 706 includes a heart rate signal generator module 754 configured to implement determine physiological information relating to a user that is wearing a wearable device. Heart rate signal generator module 754 can include a heart rate processor module 756 and an anomaly detector 758, any of which can be configured to provide one or more functions described herein.

Referring back to FIG. 1, wearable device 170 can be in communication (e.g., wired or wirelessly) with a mobile device 180, such as a mobile phone or computing device. In some cases, mobile device 180, or any networked computing device (not shown) in communication with wearable device 170 or mobile device 180, can provide at least some of the structures and/or functions of any of the features described herein. As depicted in FIG. 1 and other figures, the structures and/or functions of any of the above-described features can be implemented in software, hardware, firmware, circuitry, or any combination thereof. Note that the structures and constituent elements above, as well as their functionality, may be aggregated or combined with one or more other structures or elements. Alternatively, the elements and their functionality may be subdivided into constituent sub-elements, if any. As software, at least some of the above-described techniques may be implemented using various types of programming or formatting languages, frameworks, syntax, applications, protocols, objects, or techniques. For example, at least one of the elements depicted in FIG. 1 (or any subsequent figure) can represent one or more algorithms. Or, at least one of the elements can represent a portion of logic including a portion of hardware configured to provide constituent structures and/or functionalities.

For example, heart rate signal generator 400 of FIG. 4 and any of its one or more components, such as piezoelectric signal characterizer 432, anomalous signal filter 442, heartbeat identification determinator 434, and mask generator 444, can be implemented in one or more computing devices (i.e., any mobile computing device, such as a wearable device or mobile phone, whether worn or carried) that include one or more processors configured to execute one or more algorithms in memory. Thus, at least some of the elements in FIG. 1 (or any subsequent figure) can represent one or more algorithms. Or, at least one of the elements can represent a portion of logic including a portion of hardware configured to provide constituent structures and/or functionalities. These can be varied and are not limited to the examples or descriptions provided.

As hardware and/or firmware, the above-described structures and techniques can be implemented using various types of programming or integrated circuit design languages, including hardware description languages, such as any register transfer language (“RTL”) configured to design field-programmable gate arrays (“FPGAs”), application-specific integrated circuits (“ASICs”), multi-chip modules, or any other type of integrated circuit. For example, heart rate signal generator 400 of FIG. 4 and any of its one or more components, such as piezoelectric signal characterizer 432, anomalous signal filter 442, heartbeat identification determinator 434, and mask generator 444, can be implemented in one or more computing devices that include one or more circuits. Thus, at least one of the elements in FIG. 1 (or any subsequent figure) can represent one or more components of hardware. Or, at least one of the elements can represent a portion of logic including a portion of circuit configured to provide constituent structures and/or functionalities.

According to some embodiments, the term “circuit” can refer, for example, to any system including a number of components through which current flows to perform one or more functions, the components including discrete and complex components. Examples of discrete components include transistors, resistors, capacitors, inductors, diodes, and the like, and examples of complex components include memory, processors, analog circuits, digital circuits, and the like, including field-programmable gate arrays (“FPGAs”), application-specific integrated circuits (“ASICs”). Therefore, a circuit can include a system of electronic components and logic components (e.g., logic configured to execute instructions, such that a group of executable instructions of an algorithm, for example, and, thus, is a component of a circuit). According to some embodiments, the term “module” can refer, for example, to an algorithm or a portion thereof, and/or logic implemented in either hardware circuitry or software, or a combination thereof (i.e., a module can be implemented as a circuit). In some embodiments, algorithms and/or the memory in which the algorithms are stored are “components” of a circuit. Thus, the term “circuit” can also refer, for example, to a system of components, including algorithms. These can be varied and are not limited to the examples or descriptions provided.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. An apparatus comprising:
a skin surface microphone (“SSM”) comprising:
a piezoelectric sensor configured to receive acoustic signals and generate piezoelectric signals;
a wearable housing including the SSM, the wearable housing configured to position the SSM to receive an acoustic signal originating from human tissue; and
a physiological signal generator configured to receive a piezoelectric signal based on the acoustic signal, and the physiological signal generator being configured further to generate a physiological signal including data representing a heartbeat.

2. The apparatus of claim 1, wherein the physiological signal generator is further configured to generate the heart rate signal based on the heartbeat.

3. The apparatus of claim 2, wherein the acoustic signal is generated either by blood vessel pulsation or a human heart, or both.

4. The apparatus of claim 1, further comprising:
a heart rate processor configured to determine the heart rate; and
an anomaly detector configured to reduce anomalous portions of the acoustic signal that are anomalous to a range of acoustic characteristics associated with the heartbeat.

5. The apparatus of claim 1, wherein a portion of the wearable housing is configured to position a surface of the SSM adjacent the surface of the human tissue, a portion of the SSM being formed external to a surface of the wearable housing to contact human tissue.

6. The apparatus of claim 1, wherein a portion of the piezoelectric sensor is formed external to a surface of the wearable housing, the portion of the piezoelectric sensor being configured to contact the human tissue.

7. The apparatus of claim 1, wherein the SSM is encapsulated in the wearable housing.

8. The apparatus of claim 7, wherein the wearable housing further comprising:
an encapsulant having an acoustic impedance value in a range of acoustic impedance values including a value of acoustic impedance for the human tissue.

9. The apparatus of claim 1, wherein the SSM further comprising:
a coupler having an acoustic impedance equivalent to the human tissue, at least a first surface of the coupler being formed external to a surface of the wearable housing and second surface of the coupler being configured to communicate the acoustic signal from the first surface of the coupler to the piezoelectric sensor.

10. The apparatus of claim 1, further comprising:
a piezoelectric signal characterizer configured characterize portions of the piezoelectric signal; and
an anomalous signal filter to identify a subset of the portions of the piezoelectric signal anomalous to a range of acoustic characteristics associated with the heartbeat.

11. The apparatus of claim 10, wherein the anomalous signal filter is configured to deemphasize the subset of the portions of the piezoelectric signal based on context data.

12. The apparatus of claim 11, wherein the context data includes one or more of age data, location data, and motion data.

13. The apparatus of claim 10, further comprising:
a mask generator configured to mask time intervals in which sampling is suppressed; and
a window interval determinator configure to determine other time intervals in which the portions of the piezoelectric signal include data representing the heartbeat.

14. The apparatus of claim 1, further comprising:
a heartbeat identification determinator configured to identify the heartbeat and to identify subsequent heartbeats to determine a heart rate.

15. A method comprising:
receiving an acoustic signal originating from human tissue, the acoustic signal associated with a physiological characteristic;
generating a piezoelectric signal responsive to the acoustic signal;
determining a portion of the piezoelectric signal associated with a heartbeat derived from the acoustic signal;
identifying a heart rate at a processor based on the portion of the piezoelectric signal; and
causing generation of data representing the heart rate for presentation.

16. The method of claim 15, wherein receiving the acoustic signal originating from the human tissue comprises:
receiving the acoustic signal via a coupler configured to communicate the acoustic signal from a surface of the human tissue to a piezoelectric sensor, the coupler having an acoustic impedance equivalent to the human tissue.

17. The method of claim 15, further comprising:
deemphasizing a portion of the piezoelectric signal based on context data.

18. The method of claim 15, further comprising:
transmitting data representing the heart rate to a device.

19. A wearable device comprising:
a skin surface physiological device comprising:
a piezoelectric sensor configured to receive signals and generate piezoelectric signals;
a housing including the skin surface physiological device, the housing configured to position the skin surface physiological device to receive a signal originating from human tissue; and
a physiological signal generator configured to receive a piezoelectric signal based on the signal, and the physiological signal generator being configured further to generate a physiological signal including data representing a heartbeat.

20. The wearable device of claim 19, wherein the piezoelectric sensor comprises:
an acoustic sensor.
